(19) United States
(12) Patent Application Publication
(10) Pub. No.: US 2024/0238079 A1
(43) Pub. Date: Jul. 18, 2024

(54) CORNEAL IMPLANT WITH A RIGID RING

(71) Applicant: Alcon Inc., Fribourg (CH)

(72) Inventors: Niccolo Maschio, Winterthur (CH); Reto Grüebler, Greifensee (CH)

(21) Appl. No.: 18/413,395

(22) Filed: Jan. 16, 2024

Related U.S. Application Data
(60) Provisional application No. 63/480,405, filed on Jan. 18, 2023.

(51) Int. Cl. A61F 2/14

(52) U.S. Cl. CPC A61F 2/142; A61F 2/15

(57) ABSTRACT

In certain embodiments, a corneal implant includes a transparent dome and a ring. The transparent dome has a circular base with a perimeter that has a diameter of less than 2 centimeters. The transparent dome comprises a transparent biocompatible material. The ring has an annular shape and is disposed about the perimeter of the circular base of the transparent dome. The ring comprises a biocompatible material with a surface. The peripheral portion of the surface forms openings that facilitate attachment of eye tissue to the ring. Each opening has a diameter of less than 2 millimeters.

### TECHNICAL FIELD

The present disclosure relates generally to ophthalmic implants, and more particularly to a corneal implant with a rigid ring.

### BACKGROUND

Corneal diseases, such as keratoconus, endothelial dystrophy, and bullous keratopathy, can cause clouding and distortion of vision, and may eventually result in blindness. Surgical treatment of such diseases includes corneal transplantation. In this procedure, diseased corneal tissue is removed from the patient's eye and replaced with donor corneal tissue. However, in some cases, the patient's immune system rejects the donor corneal tissue.

### BRIEF SUMMARY

In certain embodiments, a corneal implant includes a transparent dome and a ring. The transparent dome has a circular base with a perimeter that has a diameter of less than 2 centimeters. The transparent dome comprises a transparent biocompatible material. The ring has an annular shape and is disposed about the perimeter of the circular base of the transparent dome. The ring comprises a biocompatible material with a surface. The peripheral portion of the surface forms openings that facilitate attachment of eye tissue to the ring. Each opening has a diameter of less than 2 millimeters.

Embodiments may include none, one, some, or all of the following features:
The biocompatible material comprises a biocompatible metal or a biocompatible plastic.
The openings have a density of at least 20 openings per centimeter-squared.
Each opening has a diameter of less than 1 millimeter.
The openings are arranged in a circle-packing pattern.
The openings form an artificial trabecular meshwork.
The corneal implant includes a lens disposed outwardly from the circular base of the transparent dome and substantially centered within the perimeter of the circular base of the transparent dome. An iris design may be printed on the lens.
The corneal implant includes an iris piece disposed outwardly from the circular base of the transparent dome and substantially centered within the perimeter of the circular base of the transparent dome. A lens may be disposed outwardly from the iris piece and substantially centered with the iris piece.
The corneal implant includes one or more flaps. Each flap has an attachment end that couples to a part of the corneal implant, and a suture end that can be sutured to the eye tissue. The attachment end may be coupled to the perimeter of the circular base or to the peripheral portion of the ring. A flap may have one or more drainage channels, where each channel moves fluid between the attachment end and the suture end.

In certain embodiments, a corneal implant includes a transparent dome, a ring, a lens, and one or more flaps. The transparent dome has a circular base with a perimeter that has a diameter of less than 2 centimeters. The transparent dome comprises a transparent biocompatible material. The ring has an annular shape and is disposed about the perimeter of the circular base of the transparent dome. The ring comprises a biocompatible material with a surface. The peripheral portion of the surface forms openings that facilitate attachment of eye tissue to the ring. Each opening has a diameter of less than 2 millimeters. The lens is disposed outwardly from the circular base of the transparent dome and substantially centered within the perimeter of the circular base. An iris design is printed on the lens. Each flap has an attachment end coupled to a part of the corneal implant and a suture end that can be sutured to the eye tissue.

Embodiments may include none, one, some, or all of the following features:
The attachment end is coupled to the perimeter of the circular base or to the peripheral portion of the ring.
A flap has one or more drainage channels, where each channel can move fluid between the attachment end and the suture end.

In certain embodiments, a corneal implant includes a transparent dome, a ring, an iris piece, and a lens. The transparent dome has a circular base with a perimeter that has a diameter of less than 2 centimeters. The transparent dome comprises a transparent biocompatible material. The ring has an annular shape and is disposed about the perimeter of the circular base of the transparent dome. The ring comprises a biocompatible material with a surface. The peripheral portion of the surface forms openings that facilitate attachment of eye tissue to the ring. Each opening has a diameter of less than 2 millimeters. The iris piece is disposed outwardly from the circular base of the transparent dome and is substantially centered within the perimeter of the circular base. The lens is disposed outwardly from the iris piece and is substantially centered with the iris piece.

Embodiments may include the following feature:
The corneal implant includes one or more flaps. Each flap has an attachment end, a suture end, and one or more drainage channels. The attachment end may be coupled to the perimeter of the circular base or to the peripheral portion of the ring. The suture end can be sutured to the eye tissue. Each channel can move fluid between the attachment end and the suture end.

### BRIEF DESCRIPTION OF THE DRAWINGS

FIG. 1 illustrates an example of a corneal implant that may be implanted into an eye of a patient, according to certain embodiments;

FIG. 2 illustrates an example of a corneal implant with a lens and an iris, according to certain embodiments;

FIGS. 3A and 3B illustrate an example of a corneal implant with a porous surface, according to certain embodiments;

FIGS. 4A and 4B illustrate an example of a corneal implant with one or more flaps that may be used to attach the implant to an eye, according to certain embodiments; and

FIG. 5 illustrates a diagram of an example of a method for implanting an implant into an eye, according to certain embodiments.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

Donor corneal tissue implanted into a patient's eye runs the risk of rejection by the patient's immune system. To address this issue, embodiments of a corneal implant described herein comprise biocompatible materials and include a ring with a surface that improves implantation. For example, the surface may be porous to facilitate adhesion of the implant to surrounding tissue, such as the trabecular meshwork. Additional features are described in more detail below.

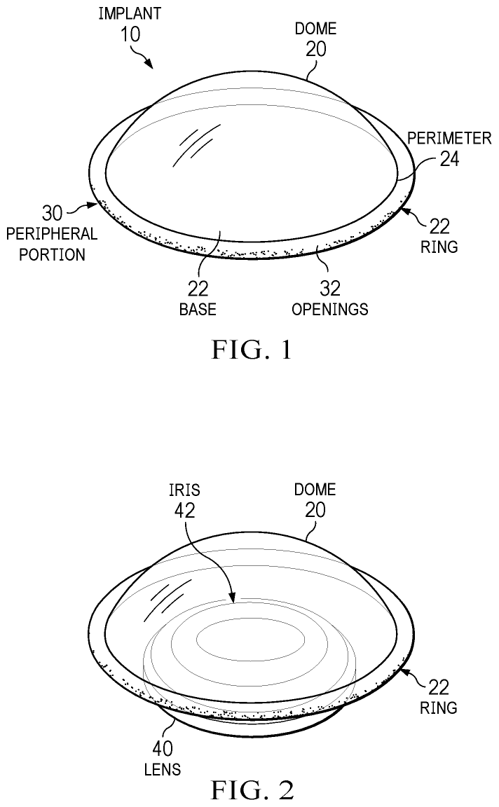

FIG. 1 illustrates an example of a corneal implant 10 that may be implanted into an eye of a patient, according to certain embodiments. In the example, implant 10 includes a transparent dome 20 and a ring 22, coupled as shown. Transparent dome 20 has a circular base 22 with a perimeter 24 having a diameter less than 2 centimeters (cm), such as in the range of, e.g., 7 to 18 millimeters (mm). Transparent dome 20 comprises a transparent biocompatible material, e.g., acrylic, silicon, or any material commonly used in an intraocular lenses (IOL).

In certain embodiments, the shape of dome 20 is determined according to the desired refractive properties of dome 20. For example, dome 20 may be required to have refractive properties of a natural cornea that provides optimal vision (or other suitable refractive properties). Given the refractive index of the material of dome 20, the shape can be calculated using, e.g., a ray-tracing technique or an intraocular lens (IOL) calculation technique.

Ring 22 has an annular shape and is disposed about perimeter 24 of circular base 22. Ring 22 comprises a rigid biocompatible material, such as a biocompatible metal (e.g., surgical steel or titanium) or a biocompatible plastic (e.g., rigid acrylic or silicone) with a high Shore hardness (e.g., Shore D value 30 or more). Peripheral portion 30 of ring 22 generally describes the outer portion of ring 22 that comes into contact with the corneal or other eye tissue. The surface of ring 22 forms openings 32, where each opening has a diameter of less than 1 or 2 millimeters. In certain embodiments, there are at least 20, 25, 50, or 100 openings 32. Openings 32 may form a porous surface or an artificial trabecular meshwork that mimics the structure of the natural trabecular meshwork of the eye.

FIG. 2 illustrates an example of a corneal implant 10 with a lens 40 and an iris 42, according to certain embodiments. In the example, lens 40 is disposed outwardly from and substantially centered within the perimeter of circular base 22 of transparent dome 20. Lens comprises a transparent biocompatible material (as described above with reference to transparent dome 20). The shape of lens 40 may designed according to the desired refractive properties of lens 40 (as described above with reference to transparent dome 20).

Iris 42 may have a size, shape, and color that matches that of the patient's other eye. Iris 42 may have any suitable form. In certain embodiments, iris 42 may be an iris design (i.e., a design that looks like a natural iris) printed on lens 40 by a printer, e.g., an inkjet printer. In other embodiments, iris 42 may be formed as a separate piece that can be disposed between lens 40 and dome 20 and coupled mechanically or chemically. In the embodiments, the iris piece may be an iris design printed on a substrate comprising a biocompatible material, such as a transparent biocompatible material.

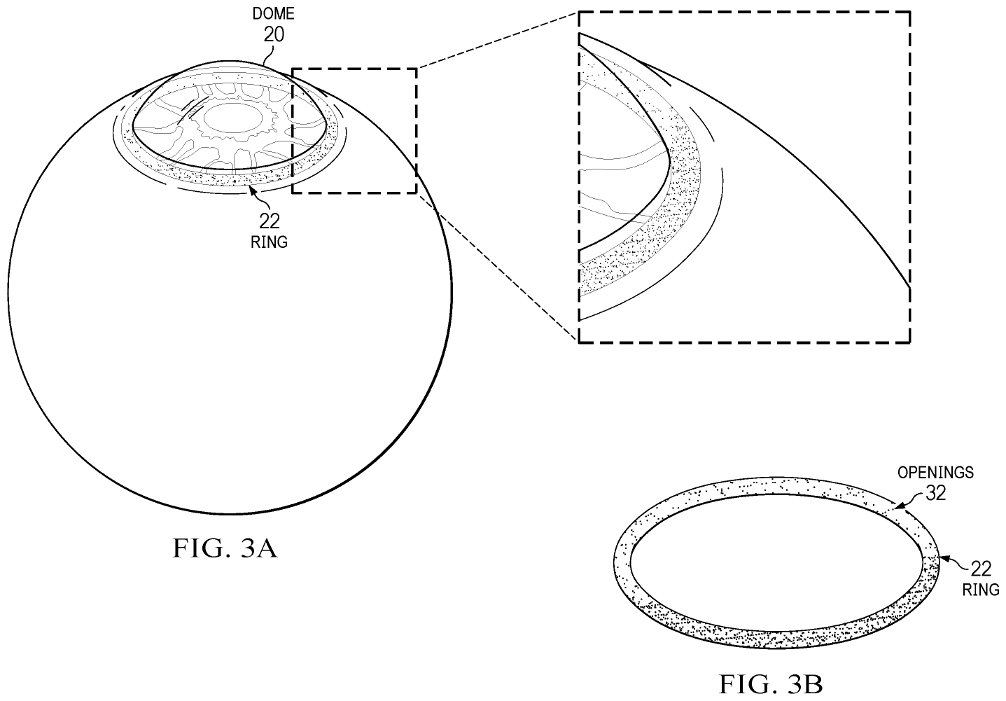

FIGS. 3A and 3B illustrate an example of a corneal implant 10 with a porous surface designed to encourage growth of surrounding tissue, according to certain embodiments. The peripheral portion (or a smaller or larger portion) of ring 22 has a surface of openings 32. An opening 32 may open into a hollow interior of ring 22 or may be a deep indentation in ring 22. Openings 32 facilitate adhesion of implant 10 to surrounding tissue, such as the natural trabecular meshwork. The natural trabecular meshwork is a complex, fenestrated, three-dimensional structure located within the iridocorneal angle. The meshwork drains aqueous humor (AH) out of the eye and operates to adjust the intraocular pressure (IOP) of the eye.

In certain embodiments, openings 32 form a porous surface with any suitable number of any suitable size of pores. For example, the pores may have a diameter of less than 1 or 2 millimeters, such as in the range of 20 to 50 microns, and a density of at least 20 pores per centimeter-squared (cm²), such as 50 to 100 pores per centimeter-squared. The pores may be arranged in any suitable pattern, such as a circle-packing pattern, e.g., triangular, square, or hexagonal packing pattern. In some embodiments, openings 32 form an artificial trabecular meshwork that mimics the structure of the natural trabecular meshwork of the eye.

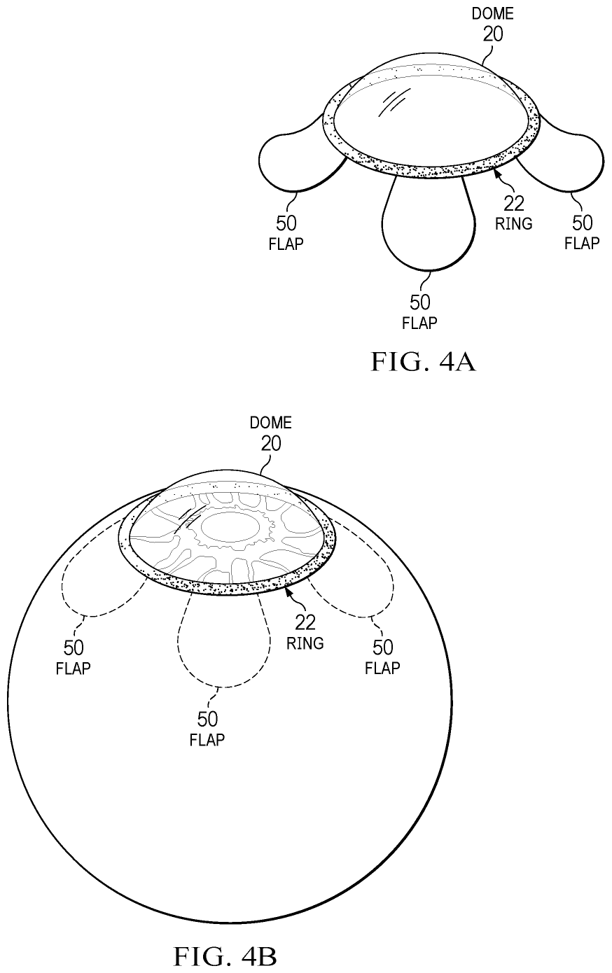

FIGS. 4A and 4B illustrate an example of a corneal implant 10 with one or more flaps 50 that may be used to attach implant 10 to the eye, according to certain embodiments. Flaps 50 may attach implant 20 to an eye in any suitable manner. For example, flaps 50 may be sutured to the eye. The sutures may be placed underneath the conjunctiva to avoid causing discomfort.

Implant 10 may have any suitable number of flaps 50, such as 1, 2, 3, 4, or more flaps 50, disposed outwardly from the perimeter of the circular base of dome 20 and/or from the peripheral portion of the ring 22. Flaps 50 may comprise a biocompatible material, such as a flexible biocompatible plastic with a Shore A hardness of 80 or less. Flaps 50 may have any suitable size and shape. For example, a flap 50 may have an attachment end shaped to conform to the part of dome 20 or ring 22 where the end is attached. Flap 50 may have a suture end shaped to avoid causing discomfort, e.g., a rounded shape. In certain embodiments, a flap 50 may have a channel between the attachment and suture ends that can drain fluid to control IOP. A flap 50 may have any suitable number of channels, e.g., 1 to 10, and the channels may have any suitable diameter, such as less than 1 or 2 millimeters.

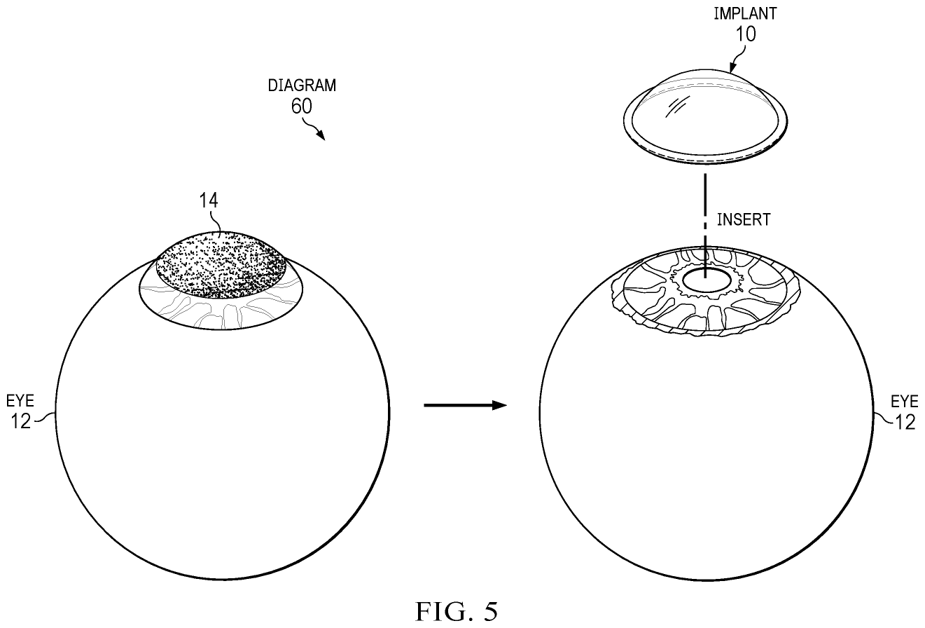

FIG. 5 illustrates a diagram 60 of an example of a method for implanting implant 10 into an eye 12, according to certain embodiments. In the example, diseased corneal tissue 14 is removed from eye 12, leaving a space in the remaining tissue. Implant 10 is inserted into the space where tissue 14 was removed. The edge of implant 10 may be placed underneath a residual ring of the remaining corneal or other eye tissue to yield a smooth transition from the sclera to the cornea. In certain embodiments, implant 10 may be sutured into place by, e.g., suturing flaps (not shown) of implant 10 to the remaining tissue. The flaps may be guided through the natural trabecular meshwork, and the sutures may be placed underneath the conjunctiva to avoid causing discomfort.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components, as apparent to those skilled in the art. As another example, modifications may be made to the methods disclosed herein. The methods may include more, fewer, or other steps, and the steps may be performed in any suitable order, as apparent to those skilled in the art.

To aid the Patent Office and readers in interpreting the claims, Applicants note that they do not intend any of the claims or claim elements to invoke 35 U.S.C. § 112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. § 112(f).

What is claimed:

1. A corneal implant comprising:
a transparent dome with a circular base, a perimeter of the circular base having a diameter of less than 2 centimeters, the transparent dome comprising a transparent biocompatible material; and
a ring having an annular shape and disposed about the perimeter of the circular base of the transparent dome, the ring comprising a biocompatible material with a surface, a peripheral portion of the surface forming a plurality of openings, each opening having a diameter of less than 2 millimeters, the plurality of openings facilitating attachment of eye tissue to the ring.

2. The corneal implant of claim 1, the biocompatible material comprising a biocompatible metal.

3. The corneal implant of claim 1, the biocompatible material comprising a biocompatible plastic.

4. The corneal implant of claim 1, the plurality of openings having a density of at least 20 openings per centimeter-squared.

5. The corneal implant of claim 1, each opening having a diameter of less than 1 millimeter.

6. The corneal implant of claim 1, the plurality of openings arranged in a circle-packing pattern.

7. The corneal implant of claim 1, the plurality of openings forming an artificial trabecular meshwork.

8. The corneal implant of claim 1, further comprising a lens disposed outwardly from the circular base of the transparent dome and substantially centered within the perimeter of the circular base of the transparent dome.

9. The corneal implant of claim 8, further comprising an iris design printed on the lens.

10. The corneal implant of claim 1, further comprising an iris piece disposed outwardly from the circular base of the transparent dome and substantially centered within the perimeter of the circular base of the transparent dome.

11. The corneal implant of claim 10, a lens disposed outwardly from the iris piece and substantially centered with the iris piece.

12. The corneal implant of claim 1, further comprising one or more flaps, each flap comprising:
an attachment end configured to couple to a part of the corneal implant; and
a suture end configured to be sutured to the eye tissue.

13. The corneal implant of claim 12, the attachment end coupled to the perimeter of the circular base.

14. The corneal implant of claim 12, the attachment end coupled to the peripheral portion of the ring.

15. The corneal implant of claim 12, a flap comprising one or more drainage channels, each channel configured to move fluid between the attachment end and the suture end.

16. A corneal implant comprising:
a transparent dome with a circular base, a perimeter of the circular base having a diameter of less than 2 centimeters, the transparent dome comprising a transparent biocompatible material;
a ring having an annular shape and disposed about the perimeter of the circular base of the transparent dome, the ring comprising a biocompatible material with a surface, a peripheral portion of the surface forming a plurality of openings, each opening having a diameter of less than 2 millimeters, the plurality of openings facilitating attachment of eye tissue to the ring;
a lens disposed outwardly from the circular base of the transparent dome and substantially centered within the perimeter of the circular base of the transparent dome, an iris design printed on the lens; and
one or more flaps, each flap comprising:
an attachment end configured to couple to a part of the corneal implant; and
a suture end configured to be sutured to the eye tissue.

17. The corneal implant of claim 16, the attachment end coupled to the perimeter of the circular base or to the peripheral portion of the ring.

18. The corneal implant of claim 16, a flap comprising one or more drainage channels, each channel configured to move fluid between the attachment end and the suture end.

19. A corneal implant comprising:
a transparent dome with a circular base, a perimeter of the circular base having a diameter of less than 2 centimeters, the transparent dome comprising a transparent biocompatible material;
a ring having an annular shape and disposed about the perimeter of the circular base of the transparent dome, the ring comprising a biocompatible material with a surface, a peripheral portion of the surface forming a plurality of openings, each opening having a diameter of less than 2 millimeters, the plurality of openings facilitating attachment of eye tissue to the ring;
an iris piece disposed outwardly from the circular base of the transparent dome and substantially centered within the perimeter of the circular base of the transparent dome; and
a lens disposed outwardly from the iris piece and substantially centered with the iris piece.

20. The corneal implant of claim 19, further comprising one or more flaps, each flap comprising:
an attachment end configured to couple to a part of the corneal implant, the attachment end coupled to the perimeter of the circular base or to the peripheral portion of the ring;
a suture end configured to be sutured to the eye tissue; and
one or more drainage channels, each channel configured to move fluid between the attachment end and the suture end.
